(19) United States
(12) Patent Application Publication
(10) Pub. No.: US 2020/0286603 A1
(43) Pub. Date: Sep. 10, 2020

(54) MOOD SENSITIVE, VOICE-ENABLED MEDICAL CONDITION COACHING FOR PATIENTS

(71) Applicant: THE BOARD OF TRUSTEES OF THE UNIVERSITY OF ILLINOIS, Urbana, IL (US)

(72) Inventors: Olusola Ajilore, Chicago, IL (US); Alex Leow, Chicago, IL (US); Philip Yu, Chicago, IL (US); Ben Gerber, Glenview, IL (US); Faraz Hussain, Chicago, IL (US); Jun Ma, Hinsdale, IL (US)

(21) Appl. No.: 16/648,711

(22) PCT Filed: Sep. 25, 2018
(86) PCT No.: PCT/US18/52581

§ 371 Date: Mar. 19, 2020

Related U.S. Application Data
(60) Provisional application No. 62/562,600, filed on Sep. 25, 2017.

(51) Int. Cl. G16H 10/60; G16H 20/10; G16H 20/60; G16H 50/70; G16H 50/20; G16H 20/70; G16H 40/67; G06N 20/00

(52) U.S. Cl. CPC G16H 10/60; G16H 20/10; G16H 20/60; G06N 20/00; G16H 50/20; G16H 20/70; G16H 40/67; G16H 50/70

(57) ABSTRACT

A method includes receiving voice data from a voice-enabled device associated with a user, the voice data indicating a request for an output, determining from the voice data a current mood and/or cognitive state of the user, determining an output in response to the request, and adjusting the output and/or the form of the output according to the determined current mood and/or cognitive state of the user.

### TECHNOLOGICAL FIELD

The present disclosure relates to technology for assisting patients in self-care related to particular disease conditions and, in particular, to voice-enabled coaching capable of providing information to patients, taking actions on behalf of patients, and providing general support of disease self-management.

### BACKGROUND

Many chronic diseases, such as diabetes, bipolar disorder, and the like require a patient to manage his or her own care on a day to day basis, with period intervention by a medical professional. Successfully managing such a condition on a day-to-day basis can be difficult when there are numerous factors to consider that can complicate, or at least affect, the steps required to properly care for oneself. Diabetes, for example, requires managing one's diet, medications, various blood levels, blood pressure, and other factors that are interrelated and that can change the necessary treatment (e.g., insulin administration) on a daily basis.

Additionally, various studies have shown that patient mood and cognitive state can have a profound affect on the physiological well-being of a patient and on the patient's ability to care for himself or herself. For example, depression in type-2 diabetes has been associated with impaired self-management behavior, worse diabetic complications, and higher rates of mortality. Additionally, diabetes-related distress contributes to worsening outcomes in type-2 diabetes. Many studies have demonstrated that techniques such as problem-solving therapy and diabetes coaching can mitigate the negative effects of depression and distress as well as improve self-management and glycemic control.

Traditionally, patients have been assisted in self-care by a variety of disparate methods that, individually, assist the patient by providing medication logging and reminders (e.g., reminding a patient to take a particular medication at a particular time), testing reminders (e.g., reminding a patient to test blood sugar levels), lifestyle suggestions (e.g., menu suggestions, exercise reminders, etc.), education, social and emotional support, and the like. Even in instances in which some or all of these functions have been integrated into a single location (e.g., a mobile device application), the information provided to the patient typically remains static, relies on generalized data for a large patient population, and provides information agnostic as to the current physiological and/or emotional state of the patient.

Further, managing certain diseases can be difficult, even at the most basic level. For a variety of reasons, people may not have access to the training, social services, medical services, or other aspects of self-care necessary to manage their disease. These reasons include economic factors such as the patient's financial means, geographic factors such as proximity to services, social factors such as stigma associated with a disease or its treatment, and the availability of services in general.

### SUMMARY OF THE DISCLOSURE

A method includes receiving voice data from a voice-enabled device associated with a user, the voice data indicating a request for an output, determining from the voice data a current mood and/or cognitive state of the user, determining an output in response to the request, and adjusting the output and/or the form of the output according to the determined current mood and/or cognitive state of the user.

In another arrangement, a method includes receiving data representative of an input from a user, processing the data to determine one or more sentiment features for the data, and processing the data to determine one or more request characteristics corresponding to the data. The method also includes processing the one or more sentiment features to (1) identify a domain-specific service module indicated by the one or more sentiment features, (2) identify a request indicated by the one or more sentiment features, and (3) output a structured representation of the identified request. The method further includes transmitting to the identified domain-specific service module the structured representation of the identified request, transmitting to the identified domain-specific service module the identified request characteristics, determining, in the domain-specific service module, from the one or more request characteristics and/or the identified request, one or more real-time user characteristics, and determining in the domain-specific service module a response to the request indicated by the one or more sentiment features based on the structured representation of the identified request and the identified real-time user characteristics, the response represented as text. The method includes processing the response to output a speech representation of the text response, and transmitting the speech representation to a voice-enabled device.

A system includes a server comprising a processor configured to execute machine readable instructions. The machine readable instructions cause the processor to receive input data from a plurality of selectively connected input sources. The input sources include at least one of the group consisting of: a device recording physiological data of a user; a device monitoring health status of the user; a mobile device associated with the user and providing environmental data and/or data related to the user's interaction with the mobile device; a source of electronic medical record data; and a device receiving voice input from the user. The instructions also cause the processor to analyze the received input data relative to prior data stored in a machine-learning algorithm database (MLAD), using one or more machine learning algorithms to determine, within a predetermined time frame, (1) a notification to send to the user and/or (2) an update to the MLAD, wherein the determination of the notification and/or the determination of the update to the MLAD is based on input data received during the predetermined time frame, data in the MLAD, and data stored in a best practices medical database.

A method comprises receiving at a server, a request from a user for information or to perform an action. The method also includes receiving at the server additional data related to the request, the additional data including at least one of the group consisting of: (1) data from a best practices medical database; (2) real-time data indicative of a mood of the user and/or a cognitive state of the user; and (3) a database storing machine-learning data related to the user and/or a set of users having in common with the user a particular characteristic. Further, the method includes analyzing in the server the request and the additional data related to the request to generate the information requested by the user or perform the action requested by the user.

In yet another method the method selectively, and in real time, generates medical notifications for delivery to a device associated with a patient. The method includes receiving, at a server, data associated with the patient. The method then includes analyzing (1) the received data, (2) a best practices medical database, and (3) a database storing machine-learning data related to the patient and/or a set of users having in common with the patient a particular medical condition. The method further includes determining from the analysis whether to generate a medical notification and transmit the medical notification to the device associated with the patient, and determining from the analysis whether to update the database.

### BRIEF DESCRIPTION OF THE DRAWINGS

The present description will be presented with reference to the drawings in which like reference numerals are used where possible to refer to like elements.

FIG. 1 is a block diagram depicting an example system including a variety of input sources as described herein.

FIG. 2 is a block diagram depicting a server in accordance with the example system of FIG. 1.

FIG. 3 is a block diagram depicting an example mobile device.

FIG. 4 is a messaging diagram illustrating message flow in an example embodiment.

FIG. 5 is a flow chart illustrating an example method according to described embodiments.

### DETAILED DESCRIPTION

The following is a description of a self-care ecosystem that addresses the problems above, as well as a variety of others. In various contemplated embodiments, the ecosystem facilitates aspects of self-care, aided by deep learning algorithms and enabled by a variety of technologies and services, in a manner that is specific to the user and the user's mental and/or cognitive state, and provides information to the user accordingly.

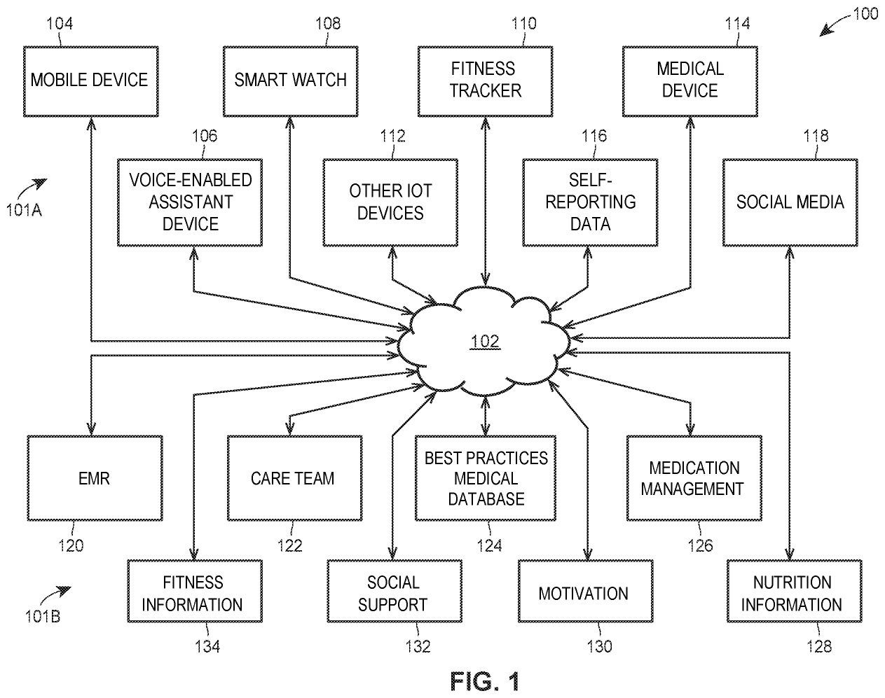

FIG. 1 depicts an example self-care ecosystem 100. The self-care ecosystem 100 includes a variety of elements that may be directly or indirectly communicatively coupled to one another via one or more networks 102, including the Internet. While depicted as a single entity in FIG. 1, the networks 102 may include interconnected networks, cloud services, and a variety of servers, databases, and other equipment (routers, switches, etc.). Additionally, some portion of the analytical capabilities associated with the self-care ecosystem 100, as well as the routines that enable interaction between the various elements coupled to one another via the networks 102, may be cloud-enabled and/or may reside on one or more servers in the one or more networks 102. Various aspects of the one or more networks will be described with respect to later figures.

As depicted in FIG. 1, the self-care ecosystem 100 may include a variety of devices and services providing data to one another and receiving data from one another, all in service of facilitating self-care for the user. The elements within the self-care ecosystem 100 other than the one or more networks 102 may be loosely divided into real-time elements 101A that provide real-time or near real-time information about the user or about the user's environment, and static elements 101B that provide information that is relatively static or, in any event, changes infrequently compared to the data received from the elements 101A.

In general, the self-care ecosystem 100 may serve different functions according to the particular embodiment, but also according to the desires of the user. The real-time elements 101A may facilitate inputs directed by the user such as requests for information, requests for action to be taken, and the like. The real-time elements 101A may also, in embodiments, passively collect data about the user, which data may be analyzed and compared to previous data for the user and/or to data of a related population (e.g., data of people having a shared characteristic such as a disease or other condition). The data may be analyzed by extracting metadata and reviewing the metadata, in embodiments. For example, keystroke data may be abstracted to keystroke metadata such as typing speed and backspace usage to determine one or more characteristics of the user's mood and/or cognitive state. The information gleaned from the analysis, and the commands or requests received from the user, may be used during interactions with static elements 101B, to request information, update records, and the like. In general, the system may provide information and/or notifications to the user in manner that is contextualized to the user's history and, in embodiments, to the user's current mood and/or cognitive states, as illustrated in the examples that follow.

The real-time elements 101A include devices carried, worn, or otherwise used by the user to provide the real-time or near real-time inputs to the self-care ecosystem 100 actively or passively. For example, as described in greater detail below, a mobile device 104 (e.g., a mobile phone, a tablet computer, a laptop, etc.) may facilitate entry of certain data by the user (i.e., active input), and may also or alternatively collect and/or analyze data related to the user's use of the mobile device (i.e., passive input). The devices may also include a voice-enabled assistant device 106. As should be recognized, the voice-enabled assistant device 106 may be a device dedicated to the voice-enabled assistant, but may also be any device that enables access to such a voice-enabled assistant and, accordingly, may include one or more of the mobile devices 104. A smart watch 108 may facilitate entry of information (i.e., active input) but, like the mobile device 104, may also collect and/or analyze data related to the user's activity, heart rate, or environment (i.e. passive input). Similarly, a fitness tracker device 110 may provide information about the user's activity level, heart rate, environment, etc. Other Internet of Things (IoT) devices 112 may likewise provide data about the user's environment and the like. In embodiments, one or more medical devices 114 may collect and/or analyze information about the user's health status and/or one or more physiological parameters such as blood glucose level, blood pressure, etc. and provide that information to other elements in the self-care ecosystem 100.

Information about the user may also enter the self-care ecosystem 100 through self-reporting mechanisms 116. For example, an application running on the mobile device 104, a website, or a question asked by voice-enabled assistant device 106 may facilitate entry by the user of his or her perceived emotional state, mood, cognitive ability, or other information that may be used to determine the user's health status, mental state, or other aspects of the user's well-being. The self-care ecosystem 100 may also be communicatively coupled to one or more social media sources 118 and, in particular, one or more social media accounts or sites on which the user is active, providing the self-care ecosystem 100 with additional data about the user's activities, environment, mood, etc.

Of course various ones of the real-time elements 101A, data may be transmitted to the server 200 via more than one method. Some of the real-time elements 101A, such as the mobile device 104, certain smart watches 108, certain medical devices 114, and voice-enabled assistant devices 106, may connect directly to the server 200 via the Internet using WiFi or mobile telephony services. Others of the real-time elements 101A, such as smart watches 108 lacking WiFi or mobile telephony capability, fitness trackers 110 lacking WiFi or mobile telephony capability, and medical devices 114 lacking WiFi or mobile telephony capability may connect to a device, such as the mobile device 104 and/or the voice-enabled assistant device 106, using a short range communication protocol such as the Bluetooth protocol, and may send data to the server 200 through the mobile device 104 or the voice-enabled assistant device 106. In still other cases, various ones of the real-time elements 101A may not communicate directly with the server 200, but may transmit data, directly or via another device, to a different server associated with another service. For example, a fitness tracker device 110 may send data to a server associated with the fitness tracking service. In such embodiments, the server 200 may access the data from the device using an application programming interface (API) for the service, as is generally understood.

The static elements 101B of the self-care ecosystem 100 include information sources that may each be user-specific, population specific, disease specific, generally applicable, public, private, subscription based, etc. For instance an electronic medical record (EMR) system 120 may include a database having electronic medical records for the user. The EMRs are, of course, user-specific and private. The EMR system 120 may receive user data from one or more of the real-time elements 101A. For example, the EMR system 120 may receive heart rate data from the smart watch 108, fitness tracker 110, or the medical device 114. As another example, if the medical device 114 is a blood glucose monitor, the EMR system 120 may receive periodic blood glucose readings from the medical device 114. In still other examples, the user may access and/or change data in his or her EMRs using an application on the mobile device 104, or an input to the voice-enabled assistant device 106.

The static elements 101B may also include a care team interface 122 that facilitates interaction between the user and a team of medical or other professionals with whom the user has a relationship. The care team interface 122 may, for example, remind the user of upcoming medical appointments, allow the user to schedule appointments, provide an interface for asking questions of various professionals related to the user's care, etc., all via the mobile device 104 and/or the voice-enabled assistant device 106 interacting with the one or more networks 102.

A best practices medical database 124 may store vetted medical advice or information. The best practices medical database 124 may be accessed by the self-care ecosystem 100 (and, as described below, particularly by various routines executing on equipment in the one or more networks 102) to provide answers to queries posed by the user, to provide advice to the user, or to guide outputs from various artificial intelligence executing in the self-care environment 100. The best practices medical database 124 may include drug information (e.g., doses, interactions, pharmacology, etc.), disease information (e.g., symptoms, causes, treatments, etc.), general health information, and any other information related to medical practice. The best practice medical database 124 need not be a single database, but could instead be multiple databases (e.g., a database storing general medical practice information and one or more databases storing disease-specific medical practice information).

A medication management module 126 may facilitate safe and consistent use of medications by a user of the self-care ecosystem 100. The medication management module 126 may keep track of medications prescribed to the user (e.g., by interacting with the EMR system 120), may monitor for possible combinations of medications that could have harmful interactions, and may assist the user in complying with the prescribed medication regimes. By way of example, a user may indicate via an app on the mobile device 104 or via a statement to the voice-enabled assistant device 106 that a particular medication has been taken, such that the medication management module 126 can log the medication dose and remind the user when it is time for the next dose. Alternatively, a network-connected pill bottle (i.e., an IoT device 112) may report to the medication management module 126 (via the network 102) that the medication contained therein has been taken. The medication management module 126 may also track consumption and fill dates of prescriptions, in embodiments, to remind the user to refill prescriptions or, in embodiments, to automatically request a refill of a prescription.

Various other modules may provide more generalized information that may contribute to the general well-being of the user. For instance, the user may, through the self-care ecosystem 100, access nutrition information 128, motivation module 130, social support module 132, and/or fitness information module 134. In so doing, the user may access healthy meal suggestions, recipes, nutritional information for particular foods, and the like from the nutrition information 128, may seek motivational stories, set and track goals, etc., with the motivation module 130, may interact with other users through the social support information 132, and may track exercise, receive fitness tips, and the like from the fitness information module 134.

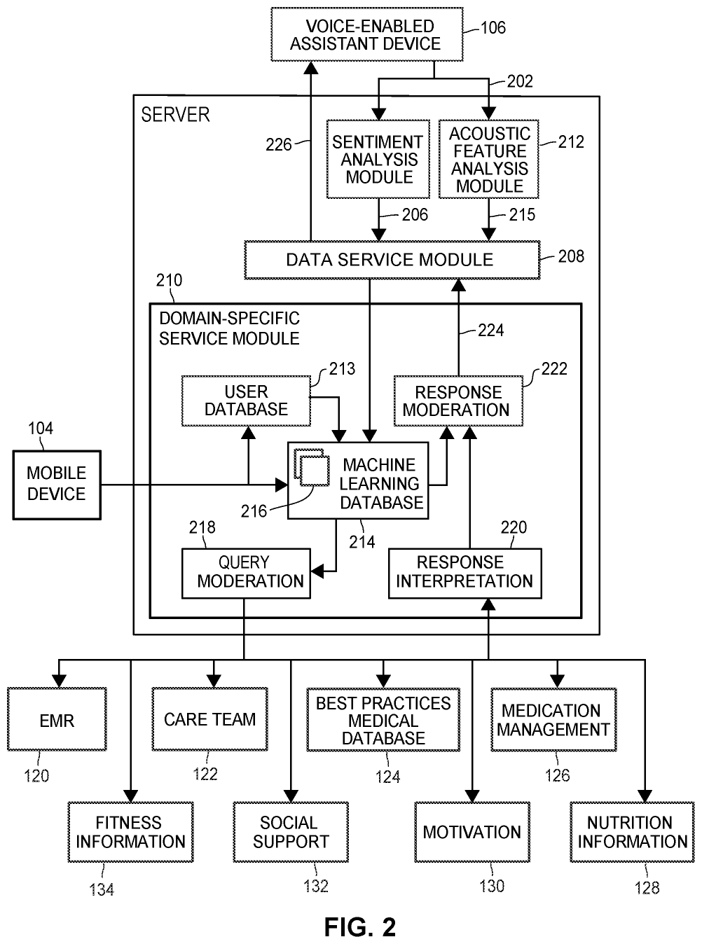

In embodiments, the one or more networks 102 include combinations of hardware and software executing to analyze data from the real-time elements 101A. In particular, the hardware and software may determine from textual, metadata, and/or acoustic (i.e., voice) input the sentiment of the user (i.e., what the user intended) and/or characteristics of the request that may be used to determine the mood of the user and/or the cognitive state of the user. Turning now to FIG. 2, the one or more networks 102 generally include one or more servers 200. The servers 200 may be dedicated servers or may be shared servers, operating as a “cloud,” as generally understood. In particular, the software executing on the one or more servers 200 may execute in a single server 200, or may be executing across multiple servers 200 for load sharing, access to data at physically disparate sites, or any of a variety of reasons that cloud services are implemented. Accordingly, throughout this description, any of the various services or software described as executing on the on the server 200 and/or on the one or more networks 102 should be understood as being executed on a single server, across multiple servers, on a cloud or distributed computing network, etc.

The server 200 receives data from the mobile device 104 and the voice-enabled assistant device 106, in embodiments. Though described in some embodiments as receiving data from both of these devices 104 and 106, it should be understood that some embodiments are contemplated in which only one or the other of the devices may be implemented. For example, in embodiments the server 200 may receive data from a mobile device 104 and not a voice-enabled assistant device 106, while in other embodiments, the server 200 may receive data from a voice-enabled assistant device 106 and not a mobile device 104. Further, even in embodiments in which the server 200 receives data from both the mobile device 104 and the voice-enabled assistant device 106, it is not required that the server 200 be receiving data from both of the devices 104 and 106 at the same time. Additionally, while a distinction is made in this description between the voice-enabled assistant device 106 and the mobile device 104, it will be understood that in some instances, the voice-enabled assistant device 106 may be integrated with the mobile device 104, such that an application executing on the mobile device 104 facilitates the user's access to the voice-enabled assistant. In these instances, the voice-enabled assistant 106 continues to operate in the manner described below, despite being resident on the mobile device 104. Stated another way, while the voice-enabled assistant device 106 is described herein as a stand-alone device, it may be integrated into other devices, including the mobile device 104, but functions in essentially the same manner regardless of the implementation.

As illustrated in FIG. 2, the voice-enabled assistant device 106 receives a vocalized request from a user and converts the physical sound to digital voice data 202 (e.g., by sampling) representative of the user request. The voice-enabled assistant device 106 transmits the voice data 202 via a network (e.g., the internet) to the server 200 and, in particular, to software routines or modules executing on the server 200. The voice data 202 may be sent to a sentiment analysis module 204 that processes the voice data 202 into text using natural language algorithms and determines the nature or meaning of the request. The sentiment analysis module 204 may also analyze the text to attempt to determine characteristics of the request (e.g., syntax, word choice, etc.) that may be used to determine the relative emotional state of the user. The sentiment analysis module 204 creates a structured representation 206 of the user's request and metadata about the request and transmits the structured representation 206 to a data service module 208. The data service module 208 may receive the representation 206 from the sentiment analysis module 204 and, from the representation 206, determine a domain-specific service module (DSSM) that is identified, either explicitly or implicitly, in the user request. For example, the data service module 208 may determine from the representation 206 that the user's request related to a particular disease condition or syndrome and may search a library of DSSMs for one that relates to the disease condition or syndrome. Alternatively, the data service module 208 may determine from the representation 206 that the user's request identified a specific DSSM (e.g., by reciting a particular word or phrase). In any event, as illustrated in FIG. 2, having identified a DSSM associated with the user's request, the data service module 208 may transmit the structured representation 206 to the identified DSSM 210.

At the same time, the voice-enabled assistant device 106 may also, in embodiments, transmit the voice data 202 via a network to an acoustic feature analysis module 212. The acoustic feature analysis module 212 may analyze the user's voice (or the sampled, digital representation of the voice) to look for vocal biomarkers and other features of the user's speech that may be used to determine the user's emotional state and/or cognitive state. For example, the acoustic feature analysis module may measure vocal characteristics such as speech volume, speech speed, presence and/or degree of slurring, speech clarity, timbre of the voice, vocal inflections, and vocal pitch. The acoustic feature analysis module 212 may output structured data 215 indicating the measured characteristics of the user's request, and may transmit that structured data 215 to the data service module 208. The data service module 208 may send the structured data 215 with the structured data 206 to the DSSM 210.

In embodiments, any potential concern about privacy—for example, because the DSSM 210 utilizes voice analysis for mood-sensing—is mitigated through the use of a word-embedding (word2vec) approach to understand the high-dimensional semantic features of verbal communication linked to mood states, which can be learned, determined, or predicted with advanced machine learning. Using word embedding, each word is mapped to a latent feature representation. Only the accumulated latent feature representations across sentences uttered during a session are stored in the system. Because the latent feature representation is not directly interpretable by human beings, the contemplated approach protects privacy of the communication. More specifically, each word may be mapped into an n-dimensional vector. The representation can then be learned, in an unsupervised manner, from any large text corpus. Privacy is protected in two steps: first, mapping from a word to a vector is not one-to-one and is internal, known only to the system; and secondly, only the accumulated feature vectors over sentences are stored, so it is not possible to figure out the original words even if the mapping were known.

These high-dimensional semantic features may be unique to the user and, as a result, may be a function of mood states of the user and, as described below, may, with machine learning algorithms be used to determine the current mood of the user.

The primary function of the DSSM 210 is to receive the user request and respond to the user request based on data accessible to the DSSM 210, from the static elements 101B, for example and, where appropriate, based on user characteristics such as the determined mood and/or cognitive state of the user. Facilitating the functionality of the DSSM 210 are a user database 213 and a machine learning database 214. The user database 213 stores data related to the user, including, in embodiments, characteristics such as name, age, home geographic location, preferred language, and/or other characteristics that remain relatively static for the user. The user database 213 may also store historical data of user mood, cognitive state, emotional state, measured vocal characteristics, and the like, received from the sentiment analysis module 204 and/or the acoustic feature analysis module 212 via the data service module 208. In so doing, the user database 213 may store a record of progressive mood and/or cognitive states of the user as determined from the requests made via the voice-enabled assistant device 106, may store a record of the vocal characteristics of the user over time, as well as a record of requests made by the user, and other data associated with the user including data received from smart watch and fitness tracker devices (e.g., fitness routine information, heart rate and rhythm information, etc.), from medical devices (e.g., glucose levels, A1c levels, blood pressure, etc.), from the user (e.g., medication dose times, self-reported mood, dietary intake, geographic location, economic status, etc.) and from other sources. Of course, much or all of the data may be time-stamped to facilitate time-based analysis of the data for patterns.

The user database 213 may store data for a specific user only, or for a group of users. It should be understood that data for multiple users may be stored in a single database while still maintaining records of which data correspond to which of the users. The database 213, for example, may be a database of other data structures, each of which stores data for a particular user. Alternatively, the server 200 may store multiple databases 212 each corresponding to a particular user.

The machine learning database 214 (also referred to herein as a machine learning algorithm database, or MLAD) is a database storing various machine learning algorithms 216 and associated data. The machine learning algorithms 216 operate to receive data from any of the data sources to which the DSSM 210 has access and to identify patterns based on the data, in order to improve the information provided to the user. For example, the machine learning algorithms 216 may operate to find relationships between mood and blood sugar levels, between cognitive state and time of day, between cognitive state and/or mood and word choice, syntax, and/or acoustic features of the user's voice. The machine learning algorithms 216 may identify patterns specific to the user by using the user's data, but may additionally or alternatively identify patterns across all users and/or across user sub-populations (i.e., across groups of users with sharing one or more specific characteristics, such as age, condition sub-type, geographic location, economic status, etc.).

The patterns identified by the machine learning algorithms 216 may be used by the MLAD 214 and, more generally, by the DSSM 210 to provide improved information to the user according to the user's mood, cognitive state, or other variables. Specifically, the MLAD 214 may receive the structured data from the data service module 208 and may analyze, in addition to the structured request, metadata related to the user's request (e.g., syntactical metadata, word choice metadata, acoustic feature data, and any data regarding or relating to the user's mood and/or cognitive state, comparing the data to patterns already identified by the machine learning algorithms 216 and stored in the MLAD 214. In doing so, the MLAD 214 may determine the mood, cognitive state, and/or other aspects of the user's well-being, and may add information to the request according to perceived or intuited needs of the user. For example, if the patterns identified by the machine learning algorithms 216 suggest that the word choice and tone of the request (as identified in the sentiment analysis module 204 and the acoustic feature analysis module 212, respectively) suggest that the user's blood sugar levels may be low (e.g., because the word choice and tone of the request are associated with a cognitive state or mood that, historically, indicates low blood sugar for the user), the MLAD 214 may provide an indication of this possibility to a query moderation module 218. The query moderation module 218 may modify the query slightly according to the data from the MLAD 214 to provide more relevant information to the user. As but one, non-limiting example, if the user requested a recipe, and the MLAD 214 determined based on syntactical and/or acoustic information that the user's blood sugar was likely low, the query moderation module 218 may specifically seek out a recipe (e.g., from the nutrition data source 128) that will safely but quickly raise the user's blood sugar. As another example, if the user requested a motivational story (e.g., from the motivation element 130), the query moderation module 218 may modify the request slightly to request a motivational story that will raise the user's mood if the MLAD 214 determines that the user may be slightly depressed.

Additionally or alternatively to forwarding the user's request to the query moderation module 218, the MLAD 214 may cause a response to the user based on the determinations made by MLAD 214. For instance, continuing with the examples above, the MLAD may cause a response to the user suggesting that the user check her blood sugar level, for the first example above or, in the second example, may cause an immediate encouraging response to the user while the motivational story is retrieved.

Still further, the MLAD 214 may additionally or alternatively update the MLAD 214, the machine learning algorithms 216, and/or the user database 213 according to the new information received. For instance, the MLAD 214 may determine that the combination of acoustic characteristics and other real-time data indicate a change in user mood or cognitive state, and may update the database 213, the algorithms 216, or the MLAD 214 with the new information, with new patterns identified by the relationship, or merely to continue the machine learning process.

In any event, a response interpretation module 220 may receive responses from any of the static elements 101B (e.g., from elements 120-134) queried by the query moderation module 218. The response interpretation module 220 may receive the response and forward the response to a response moderation module 222 that, based on data from the MLAD 214—including data of the user's mood or cognitive state—may adjust the language of the response accordingly. For example, a response may be adjusted from a declarative tone (e.g., “you should . . . ”) to a suggestive tone (e.g., “it might help to . . . ”) according to the mood or cognitive state of the user.

A response output 224 from the response moderation module 222 is transmitted to the data service module 208, which converts the textual response from the response moderation module 222 to speech data 226 that is streamed or otherwise transmitted to the voice-enabled assistant device 106 to be output as speech to the user.

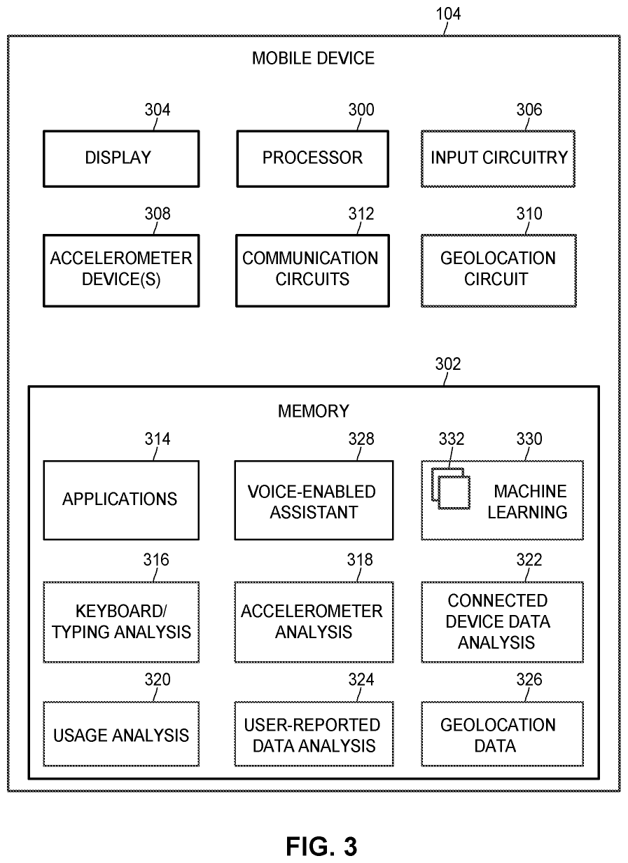

The DSSM 210 may also receive data from the mobile device 104. FIG. 3 depicts an example mobile device 104. As generally understood, the mobile device 104 includes a processor 300 coupled to a memory device 302 and configured to execute machine-readable instructions stored as applications or software on the memory device 302. The mobile device 104 also includes a display 304 and input circuitry 306. As is well known, the input circuitry 306 may include one or more buttons (not shown), one or more physical keyboards, and may also include the display 304, as is common in devices with touch-sensitive displays. While the input circuitry 306 may include a physical keyboard, it is not required, as many mobile devices have opted instead for “soft keyboards” that are displayed on the display 304 and on which the user enters input via the touch-sensitive display. The mobile device 104 also includes one or more accelerometer devices 308 that enable detection of the orientation of the mobile device 104, and a geolocation circuit 310 (e.g., a Global Positioning System receiver, a GLONASS receiver, etc.) that receives radio-frequency signals to determine the user's geographic location.

The mobile device 104 may also include a variety of communication circuits 312, each accompanied by corresponding software and/or firmware controlling the communication circuits 312. Generally, the mobile device 104 may include a cellular telephony transceiver for communicating with cellular infrastructure and providing mobile data and voice services. The communication circuits 312 may also include a transceiver configured to communicate implementing one of the family of communication protocols in the IEEE 802.11 family of protocols, commonly referred to as WiFi. The mobile device 104 may communicate with the server 200 using one or both of the cellular telephony transceiver and the WiFi transceiver, each of which is configured to communicate data from the mobile device to other devices via the Internet. As will be understood, one or both of the cellular telephony transceiver and the WiFi transceiver may also implement communication with other servers and devices via the internet.

The mobile device 104 may also communicate with other mobile devices via the WiFi transceiver. Such other devices include any WiFi-enabled device, including, in embodiments, the medical device(s) 114, the smart watch 108, the voice-enabled assistant device 106, other IoT devices 112, and of course various social media platforms, websites, and the like.

The communication circuits 312 may also include a Bluetooth-enabled transceiver communicating with one or more devices via the Bluetooth short-range communication protocol. As is generally understood, devices such as the smart watch 108, the fitness tracker 110, the medical device 114, the voice-enabled assistant device 106, and other IoT devices 112 may each, in embodiments, be configured to communicate with the mobile device 104 using the Bluetooth-enabled transceiver.

The memory 302 may store any number of general applications 314 as is common with mobile devices. However, in the mobile device 104 of the contemplated embodiments, at least one of the applications includes a specialized keyboard and typing analysis application 316. The keyboard and typing analysis application 316 may replace the default keyboard present in the operating system of the mobile device 104, and provide a different, but generally similar software keyboard through which the user may enter text into the mobile device 104. The keyboard and typing analysis application 316 may, for example, be used by the user when typing text messages (e.g., SMS message), composing electronic mail and other messages, posting to social media, and the like.

Unlike the default keyboard present in the operating system of the mobile device 104, the keyboard and typing analysis application 316 may analyze the typing characteristics of the user and generate metadata related to the user's typing. Though any number of metadata features may be generated in regard to the user's typing, some exemplary metadata features may include: interkey delay, typing speed, keypress duration, distance from last key along two axes, frequency of spacebar usage, frequency of backspace usage, frequency of autocorrect function usage, and session length. The table below defines some of these metadata features, and is intended to be exemplary, rather than limiting:

| Metadata feature | Definition |
|---|---|
| Session length | Length of typing without exceeding a predetermined |
| | delay (e.g., 5 seconds) between characters |
| Average session | Length of sessions averaged over a predetermined |
| length | period (e.g., one week) |
| Interkey delay | Time between keystrokes |
| Average interkey | Average time between keystrokes measured over a |
| delay | predetermined period (e.g., one session or one hour) |
| Keypress duration | Length of time key is pressed |
| Average keypress | Average keypress duration measured over a pre- |
| duration | determined period (e.g., one session or one hour) |
| Distance between | The distance between consecutive keys, measured |
| consecutive keys | along two axes |
| Ratio of interkey | Per-key or average ratio of the interkey delay and |
| delay to distance | the distance between keys |
| Spacebar ratio | Ratio of spacebar keypresses to total keypresses |
| Backspace ratio | Ratio of backspace keypresses to total keypresses |
| Autocorrect ratio | Ratio of autocorrect events to spacebar keypresses |
| | (or to total keypresses) |
| Circadian baseline | The cosine-based similarity between the hourly |
| similarity | distribution of keypresses/week and the hourly |
| | distribution for the period of interest |
| Metadata feature | The variability of any one or more of the metrics |
| variability | above |

The memory 302 of the mobile device 104 may also, in some embodiments, include an accelerometer analysis module 318 configured to receive information from the accelerometer device 308 and generate metadata of the accelerometer data. For instance, the accelerometer analysis module 318 may monitor the orientation of the mobile device 104 and generate the an average displacement indicating the average position of the mobile device 104 over a period of time. As an example, the average displacement may be calculated as the square root of the sum of squares of displacement along each coordinate (x, y, z) averaged over an hour, a day, a week, etc. In embodiments, the accelerometer analysis module 318 is active only when the user is typing on the keyboard. Additionally, the accelerometer analysis module 318 may be integrated with the keyboard and typing analysis module 316, in embodiments.

Various other routines or modules may perform analysis of other aspects of the mobile device 104 and its usage. For instance, a usage analysis module 320 may monitor the frequency that the user engages with the mobile device 104 and/or the times of day and days of week that the user engages with the mobile device 104. A connected device data analysis module 322 may analyze data from one or more devices coupled to the mobile device 104 via the communication circuits 312, such as the smart watch 108, the fitness tracker 110, or the medical device 114. An application or module 324 may provide an interface through which the user of the mobile device 104 may input user-reported information such as the user's perceived mood or perceived cognitive state (which is differentiated, for the purposes of this disclosure, from the mood and/or cognitive state determined by the modules executing on the server 200). A module 326 may record geolocation data or, for privacy reasons, may provide geolocation metadata, such as indicating how much time the user spends in various locations, without keeping track of the precise locations themselves.

The memory device 302 of the mobile device 104 may also store a voice-enabled assistant application 328 that provides functionality the same as or similar to the that provided by the voice-enabled assistant device 106 described above.

In some embodiments the analysis data generated by the various applications and modules 316-326 is transmitted from the mobile device 104 to the server 200 and, in particular, to the machine learning database 214 for analysis by the machine learning algorithms 216. The machine learning algorithms 216 may look for patterns in the data received from the mobile device (e.g., patterns in the keyboard metadata, in the accelerometer metadata, etc.) to determine relationships between the data (as well as other data such as data from the EMR element 120, the medication management element 126, etc.), and the user's mood and/or cognitive state.

In other embodiments, the mobile device 104 may also include have stored on the memory 302 a machine learning module 330 storing machine learning algorithms 332 that, when executed by the processor 300, can perform some of the machine learning functions described above with respect to the machine learning algorithms 216. In particular, in embodiments, the machine learning algorithms 332 on mobile device 104 perform user-specific pattern identification, while the machine learning algorithms 216 executing on the server 200 perform population-wide pattern identification among the entire population of users of the DSSM 210 or among one or more sub-populations.

Regardless of whether the data generated at the mobile device 104 is analyzed locally by the machine learning algorithms 332 in the machine learning module 330 or remotely by the machine learning algorithms 216 in the server 200, the machine learning algorithms 216, 332 may operate in accordance with known principles of machine learning. For example, the machine learning algorithms 216, 332 may analyze the keyboard metadata generated by the keyboard and typing analysis module 316 according to the methods described in Cao et al., DeepMood: Modeling Mobile Phone Typing Dynamics for Mood Detection, presented at KDD 2017, Aug. 13-17, 2017, pp. 747-755, and in Sun et al., Sequential Keystroke Behavioral Biometrics for Mobile User Identification via Multi-view Deep Learning, arXiv:1711.02703v2 [cs.CR], Nov. 14, 2017. Of course, other algorithms may be implemented additionally or alternatively to learn to identify patterns in the keyboard metadata.

The presently described embodiments contemplate those in which no voice-enabled assistant device 106 is present, and in which the server 200 receives data only from the mobile device 104. In such embodiments, the DSSM 210 may monitor the mood or cognitive state of the user and may take actions accordingly, such as providing notifications and reminders as described throughout the disclosure. While notifications and reminders are contemplated in some embodiments as synthesized vocalizations by the voice-enabled assistant device 106 when one is present, active, and implemented, notifications and reminders may take the form of text messages, notifications pushed to the mobile device 104, and the like, regardless of whether a voice-enabled assistant device 106 is active, available, or implemented.

In addition to data from the mobile device 104 and the voice-enabled assistant device 106, the machine learning algorithms 216 may make determinations of user mood and/or cognitive state based on other information available to the DSSM 210, with or without the keyboard metadata and/or the data from the voice-enabled assistant device 106. For example, in embodiments, the DSSM 210 retrieves text data posted by the user on his social media account 118, and may analyze that as well using the machine learning algorithms 216. In embodiments the data from the social media account 118 may be parsed and analyzed by the sentiment analysis module 204 before being passed by the data service module 208 to the DSSM 210. Use of data from the social media account 118 may augment the DSSM's 210 ability to determine the current mood and cognitive state of the user.

In embodiments, the DSSM 210 may determine, based on a determination that the user's mood is somewhat depressed or that the user's cognitive state is impaired in some way, that the DSSM 210 should provide increased support to the user. For example, when the user's mood or cognitive state is abnormal (or not nominal) the DSSM 210 may be programmed to send notifications to the user that it is time to take her medication(s) because the user is more likely to forget. In embodiments, the DSSM 210 may be programmed to send support notifications to the user, reminding the user to engage in mindfulness exercises, offering various ideas for mindfulness exercises, and/or sharing supportive messages from other users. In embodiments, the DSSM 210 learns over time which of the various types of notifications and support the user needs in various situations according to her mood and/or cognitive state.

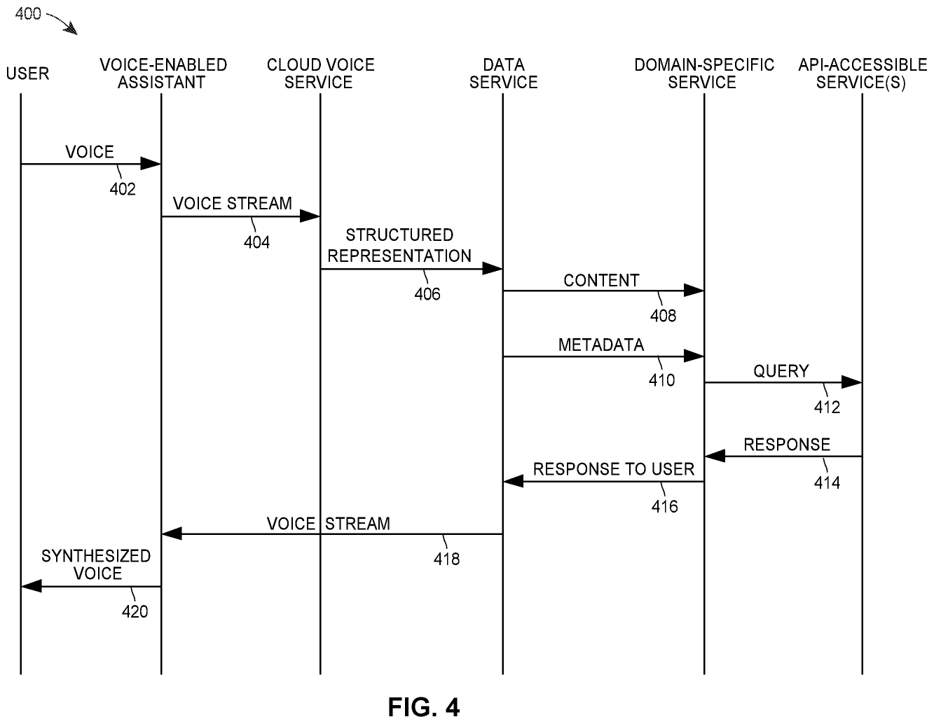

FIG. 4 is a messaging diagram 400 illustrating the communication between various elements in an embodiment. While not explicitly part of the system or method, the user may initiate a process by speaking (i.e., transmitting a voice message by generating sound waves (402)) to the voice-enabled assistant device 106. The voice-enabled assistant device 106 receives the voice message 402 and converts the sound waves into a digital representation through a sampling process, as generally understood. The digital representation is transmitted as a voice stream 404 to one or more cloud voice service modules 204, 212. The digital representation is parsed by the voice services 204, 212 to determine the content of the user's message (i.e., the user's request), as well as, in various embodiments, determine metadata associated with the syntax, word choice, and acoustic features of the user's message. The data from the voice service modules 204, 212 is output as one or more structured representations 406 (e.g., a single structured representation of the content and metadata—such as the content of the message and the syntactical and word-choice metadata—or multiple structured representations of the content, textual metadata, and acoustic metadata, respectively—such as when acoustic feature analysis data are generated by a separate module), and transmitted to the data service module 208. The data service module 208 parses the structured representation(s) 406, identifies the DSSM 210, and outputs, in embodiments, a structured representation 408 of the message content and a structured representation 410 of metadata of syntax, word choice, and/or acoustic information to the DSSM 210.

The DSSM 210 receives the content data 408 and the metadata 410, determines what data may be necessary in order to respond to the user, as well as the mood and/or cognitive state of the user, and may, where necessary, formulate one or more queries 412 to API-accessible services (e.g., the EMR data 120, the best practices medical database 124, the medication management module 126, etc.). The DSSM 210 receives a response 414 from the API-accessible service(s), and prepares the response to the user according to the user's mood and/or cognitive state, according to the response received from the API-accessible service(s), and according to the user's request. The response to the user is output as a text-based message 416 and transmitted to the data service module 208. The data service module 208 converts the text-based response into synthesized voice stream data 418, and transmits the data of the synthesized voice stream to the voice-enabled assistant device 106. The voice-enabled assistant device 106 converts the voice stream data to vibrations of a speaker, producing a synthesized voice 420 that is heard by the user.

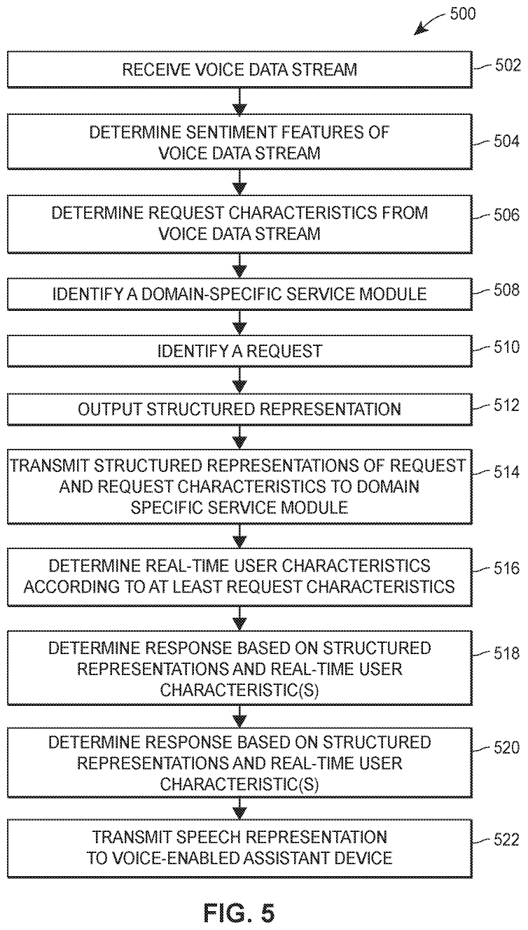

FIG. 5 is a flow diagram showing the various steps associated with an embodiment of a method 500. The server 200 receives a voice data stream (block 502) from the voice-enabled assistant device 106. One or more sentiment features of the voice data stream are determined (block 504) (e.g., by converting the voice data stream to text), for example in the sentiment analysis module 204. One or more request characteristics are determined from the voice data stream (block 506), for example by analyzing the syntax or word choice (e.g., in the sentiment analysis module 204) and/or by analyzing the acoustic features of the voice data stream (e.g., in the acoustic features analysis module 212). The domain-specific service module 210 is identified based on the sentiment features (block 508), a request is identified (block 510), and the request and request characteristics are output as a structured representation (block 512) and transmitted to the identified DSSM 210 (block 514). The DSSM 210 determines one or more real-time user characteristics according to the request and/or to the request characteristics (block 516) and determines a response based on the structured representation and the real-time user characteristic (block 518). The response is processed to output a synthesized speech representation of the response (block 520) that is transmitted to the voice-enabled assistant device (block 522).

Example Domain Specific Service Module for Diabetic Users

Depression in diabetes patients is known to be associated with impaired self-management behavior, worse diabetic complications, and higher mortality rates. Additionally, diabetes-related distress contributes to worsening outcomes, especially in type 2 diabetes. Studies have demonstrated that techniques such as problem-solving therapy and diabetes coaching can mitigate the negative effects of depression and distress as well as improve self-management and glycemic control.

An exemplary DSSM module for diabetic users is sensitive and responsive to the patient's mood and/or cognitive state. It is designed to assist patients with diabetes and, in embodiments, especially patients that are newly-diagnosed with type 2 diabetes. Given the issues of diabetes-related stress and the negative impact of depressive symptoms on type 2 diabetes, the exemplary DSSM and overall system provide solutions to help manage diabetes in the context of patients' moods and lifestyle. The exemplary DSSM for diabetic users incorporates evidence-based methods for providing patients with context-specific diabetes education, guidance, and support related to the domains of social support, lifestyle, and care coordination. The DSSM for diabetic users is designed to facilitate users' maintenance of optimal health, embracing diabetes, and the associated self-care responsibilities. Because of the functionality described above, the DSSM for diabetic users focuses on the whole person and on full health (physical, social and emotional), and is not disease centric. The DSSM for diabetic users infuses the voice-enabled assistant device and its backbone technologies with the ability to facilitate people's health and quality of life, in particular people inflicted with chronic conditions such as diabetes.

Among the functions provided by the exemplary DSSM for diabetic users are a variety of functions that for assisting diabetic users as they navigate their daily activities. In embodiments, these functions include one or more of: diabetes education, social support, medication reminders, healthy eating information, physical activity information, mindfulness information, and care coordination. As described above, the DSSM 210 may moderate each of the functions based on determinations of the user's mood and/or cognitive state. For example, the DSSM 210 may increase social support of the user through sharing and engagement of social network posts, stories, and vignettes to help the user feel supported and/or socially engaged. The DSSM 210 may suggest mindfulness exercises or counseling for stress, anxiety, or depression. In embodiments, the DSSM 210 may create virtual goals and achievements toward learning about diabetes, for taking positive steps toward self-management, for increasing healthy behaviors, etc. When needed, for example when the user's physical or mental health appears to be worsening, the DSSM 210 may engage the user's healthcare team and/or contact primary contacts designated by the user. The DSSM 210 may also provide medication dose reminders, medication refill reminders, and/or may order refills of medications.

One of the important functions of the exemplary DSSM 210 for diabetic users is diabetes education. A number of individuals newly diagnosed with diabetes often lack an understanding of metrics associated with the condition—such as hemoglobin A1c and glucose—and their desirable ranges. In embodiments, the DSSM for diabetic users can answer questions related to users' indicator levels and other concerns such as symptoms, medications and side effects—and contextualize those answers in a personalized, engaging and conversational fashion. The DSSM for diabetic users operates in accordance with the described system of FIG. 2. Specifically, a user may make an education-related request via the voice-enabled assistant device 106. The request may be specific to the user or general. For instance, the user may ask questions such as: “What is the normal range for A1c?” “Is my A1c of 6.8 too high?” “Is my blood pressure normal?” “Can itchiness be a symptom of diabetes?” “What are the side effects of Amaryl?” “How can I improve my blood glucose stability?”

Whatever the request, the voice data 202 are transmitted from the voice-enabled assistant device 106 to the server 200. At a minimum, the voice data 202 are analyzed by the sentiment analysis module 204 to determine the meaning of the request. Optionally, the sentiment analysis module 204 and/or the acoustic feature analysis module 212 may analyze the request to determine the mood and/or cognitive state of the user. In any event, the structured request is transmitted to the DSSM 210. The DSSM 210 may query various ones of the static elements 101B to determine a response to the user's request. For instance, the DSSM 210 may cause a query of the best practices medical database 124 to determine the normal range for A1c. Similarly, the DSSM 210 may query the user's electronic medical records 120 to determine the user's most recent blood pressure measurement, and may query the best practices medical database 124 to determine if that measurement is in the normal range. The DSSM 210 may also use data stored in the user database 213 to respond to queries. For example, if the user tracks meals using the system, the DSSM 210 may query the user database 213 to look up any data related to recent meals, and may query the nutrition data element 128 and/or the best practices medical database 124 and/or the user's electronic medical records 120 to compare the types of foods consumed by the user, the user's health history, nutritional data, and best practices data to suggest ways that the user could improve his management of the diabetes through control of his diet.

The DSSM for diabetic users, in embodiments, can more broadly educate users by means of a specially designed curriculum that provides bite-sized information clinically proven to improve the efficacy of diabetes self-management in response to general queries. All of these responses may be supplemented, where video content can be displayed, with video content, in embodiments. All of the responses may also be moderated according to the user's mood or cognitive state. For example, the DSSM 210 may provide a response with an emotional education component that may, for example, make the user aware of the ways that mood can impact diabetes health and the ways that diabetes health can impact mood and cognitive state.

Another function of the DSSM 210 for diabetic users is social support. When people first find out that they have diabetes, they often feel alone. As a source of support, the exemplary DSSM for diabetic users can share stories of others that have diabetes yet successfully manage the condition and overcome common barriers. The DSSM for diabetic users, in embodiments, shares a brief (e.g., 60-second) story or vignette that may provide social support and help people model adaptive behaviors. These types of stories and vignettes may be retrieved by the DSSM 210 from the motivation element 130 or the social support element 132, for example, in response to a user request to “share a success story.” Additionally, the DSSM 210 may moderate the response to the request, for example, by sharing a success story appropriate for the user's mood. The DSSM 210 may select a response that demonstrates a person overcoming a similar emotional setback as the user, for instance.

The DSSM 210 may also facilitate participation in various groups, allowing the user to communicate indirectly with other users of the DSSM for diabetic users by sending and receiving messages, posting user status messages, facilitating participation in motivational activities and challenges, and the like. For example, the user may ask the voice-enabled assistant device 106 to ask how another user (e.g., referring to the user's username) is doing (e.g., “How is John123 doing?”), may tell the voice-enabled assistant device 106 to send a motivational message to another user (e.g., “Send a cheer to John123.”), may receive similar motivational messages from others (e.g., the device 106 may say “John123 sent you a cheer.”) When the DSSM 210 detects that the user is experiencing a depressed mood, the DSSM 210 may take the opportunity to share with the user recent positive interactions (e.g., “cheers”) send to her from other users, or may suggest interactions with other users in order to facilitate a feeling of connection with others.

The DSSM 210 for diabetic users may also provide medication management functions serving, in effect, as an electronic pillbox. The DSSM 210 may remind a user to take her medication(s) and/or insulin doses. Additionally, the DSSM 210 may keep track of when a dose is administered, what the dose was, when the next dose is required, etc. If the DSSM 210 is receiving information from a medical device such as a monitor that is tracking the user's glucose and/or insulin levels, the DSSM 210 may provide additional information to alert the user when a dose of insulin or glucogon is required, or may simply notify the user, via the voice-enabled assistant device 106 or the mobile device 104, that a monitored level (e.g., glucose) is outside of the nominal range. In embodiments, the DSSM 210 for diabetic users will provide notification (via the voice-enabled assistant 106 or the mobile device 104) of when the user needs to order a refill from the pharmacy and, in embodiments, can order the refill on behalf of the user. The DSSM 210 may also respond to user requests such as: “When is my next insulin shot?” “How much metformin do I need to take?” “When do I need to refill my Glucotrol prescription?” etc. As with other aspects of the DSSM 210 function, the DSSM 210 may, in these instances, too, moderate the response to the user based on the user's mood. As an example, if the DSSM 210 detects user's mood is depressed, the DSSM 210 may respond to a request for information about the next refill by complimenting the user on his compliance with the medication regime (e.g., “Your should order your next refill by Tuesday. You've done a great job taking your medication; you haven't missed a single dose!”).

In embodiments, the DSSM 210 for diabetic users can facilitate healthy eating habits by providing healthy recipes on demand based on groceries that user reports having available in his kitchen. For example, the user may request a healthy recipe (e.g., “What is a healthy recipe with chicken and squash?”). The DSSM 210 may query the nutrition data element 128 for healthy recipes having chicken and squash among the ingredients. In embodiments, the DSSM 210 may cooperate with other DSSMs in the system to provide additional services to the user, such as ordering missing ingredients from an online grocery provider. In addition, the DSSM 210 can query a database (e.g., the nutrition data element 128) for healthy meal options to make recommendations that the user can then order though restaurant delivery services like EatStreet, Seamless and delivery.com, or using third-party meal kit services such as Blue Apron, HelloFresh or Home Chef. The user can then share her dietary choices with her social networks and followers. Still further, the user may ask questions when dining out, to get healthy options at particular dining venue (e.g., “What healthy options are there at Lucky's Restaurant?”). The DSSM 210 may query a menu of the dining venue (by interacting with other DSSMs and or search engines) and compare the menu items to information in the nutrition data element 128, before making suggestions to the user. Depending on the user's mood, the DSSM 210 may recommend a slightly less healthy option if the user appears slightly depressed, while sticking to strictly healthy options if the user's mood appears normal or upbeat. Of course, the particular recommendation may depend on other factors, as well, such as the user's fitness regime, recent blood glucose history, and the like.

The DSSM 210 for diabetic users can also help users manage their physical activity and fitness. Data regarding the user's activity can be entered manually via an application executing on the mobile device 104, can be provided from a wearable device such as the fitness tracker 110 or the smart watch 108, or other health-tracking platforms communicatively coupled to the DSSM 210. The DSSM 210 can tell the user how much activity she has already accomplished and serve as a cheerleader—as well as relay encouragement from other users of the skill that an individual has chosen to follow or connect with—to help her achieve her daily and weekly diet and exercise goals. These data may be stored in the user database 213, and information about fitness generally may be retrieved from the fitness data element 134. The user may make requests such as: “How many steps have I taken today?” “How many calories have I burned?” “Give me a 10-minute guided aerobic exercise.” “Queue up my favorite yoga routine.” When the DSSM 210 detects that the user is depressed, the DSSM 210 may moderate the response to the user, as in other instances described above. In response to a query about the number of steps taken (or even without being queried, in embodiments), the DSSM 210 may cause a notification to the user that she is on track or even doing better than her normal amount of steps, as a means of providing encouragement or positive news, for example.

In embodiments, a user can also invoke the DSSM 210 to access voice-guided physical, meditation, and relaxation exercises. This may be particularly relevant depending on mood and distress level, which, as described above, may be determined by the DSSM 210 based on the user's syntax, word choice, tone, and other acoustic indicators in their voice. In embodiments, the user need not even request these types of mindfulness exercises but, rather, the DSSM 210 may recommend them automatically when the DSSM 210 determines that the user's mood could benefit from them.

The DSSM 210 may also interface with the user's health care team (and digital services related to the health care team, such as the care team element 122), in embodiments. The health care team may include a primary care doctor, nurse, diabetes educator, ophthalmologist, podiatrist, pharmacist, psychotherapist, psychiatrist, and/or counselor. Coordinating care between these disparate team members is sometimes a significant challenge for those newly diagnosed with diabetes. The DSSM 210 for diabetic users can assist with appointment scheduling and reminders by interacting with the care team element 122 (e.g., a website, server, or API associated with one or more members of the care team), can send messages to members of the care team, etc.

Additionally or alternatively, the DSSM 210 may update and/or access electronic medical records 120 (EMRs) for the user, in some embodiments. It is important to note that, at least in some contemplated embodiments, official EMRs may not be updated, but rather the official EMRs may be supplemented with data from the DSSM 210 itself and/or data supplied by other elements of the system via the DSSM 210. The supplemental data in the official EMRs may be collected in a segregated area of the EMR database to prevent corruption of the official patient medical records, in embodiments. In some embodiments, the DSSM 210 may have read access to the official EMR information in order to provide services and information to the user, while in other embodiments, the DSSM 210 may not have any access to the official EMR information, but rather may only read and/or write to EMR information that is segregated from the official records. In any event, the DSSM 210 may write to the EMR information 120 a variety of types of data including, without limitation: data received from the medical device 114 (e.g., blood glucose measurements, blood pressure measurements), data received from the smart watch 108 and/or the fitness tracker 110 (e.g., heart rate, heart rhythm, physical activity); self-reported data 116 (e.g., user mood, dietary information, blood glucose measurements); and data gleaned from requests made by the voice-enabled assistant device 106 or from the mobile device 104 through analysis by the machine learning algorithms 216. Similarly, the data in the EMRs 120 may be available to the DSSM 210 in order to answer queries from the user (e.g., “What was my blood pressure during my last doctor visit?” “Which medication did my doctor change the dose for during my last visit?” “When was my blood glucose last above 90?”).

Throughout this specification, plural instances may implement components, operations, or structures described as a single instance. Although individual operations of one or more methods are illustrated and described as separate operations, one or more of the individual operations may be performed concurrently and, aside from prerequisite data flow, nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of components, modules, or mechanisms. Modules may constitute either software modules (e.g., code embodied on a machine-readable medium or in a transmission signal) or hardware modules. A hardware module is tangible unit capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations. Though the application describes processors coupled to memory devices storing routines, any such processor/memory device pairing may instead be implemented by dedicated hardware permanently (as in an ASIC) or semi-permanently (as in an FPGA) programmed to perform the routines.

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The one or more processors may also operate to support performance of the relevant operations in a “cloud computing” environment or as a “software as a service” (SaaS). For example, at least some of the operations may be performed by a group of computers (as examples of machines including processors), these operations being accessible via a network (e.g., the Internet) and via one or more appropriate interfaces (e.g., application program interfaces (APIs).)

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Some portions of this specification are presented in terms of algorithms performing operations on data stored as bits or binary digital signals within a machine memory (e.g., a computer memory). As used herein, an “algorithm” is a self-consistent sequence of operations or similar processing leading to a desired result. In this context, algorithms and operations involve physical manipulation of physical quantities. Typically, but not necessarily, such quantities may take the form of electrical, magnetic, or optical signals capable of being stored, accessed, transferred, combined, compared, or otherwise manipulated by a machine. It is convenient at times, principally for reasons of common usage, to refer to such signals using words such as “data,” “content,” “bits,” “values,” “elements,” “symbols,” “characters,” “terms,” “numbers,” “numerals,” or the like. These words, however, are merely convenient labels and are to be associated with appropriate physical quantities.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description should be read to include one or at least one and the singular also includes the plural unless it is obvious that it is meant otherwise.

Still further, the figures depict preferred embodiments of a system for purposes of illustration only. One skilled in the art will readily recognize from the description that alternative embodiments of the structures and methods illustrated herein may be employed without departing from the principles described herein

Upon reading this disclosure, those of skill in the art will appreciate still additional alternative structural and functional designs for a system and a process for identifying terminal road segments through the disclosed principles herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

The following list of aspects reflects a variety of the embodiments explicitly contemplated by the present disclosure. Those of ordinary skill in the art will readily appreciate that the aspects below are neither limiting of the embodiments disclosed herein, nor exhaustive of all of the embodiments conceivable from the disclosure above, but are instead meant to be exemplary in nature.

1. A method comprising: receiving voice data from a voice-enabled device associated with a user, the voice data indicating a request for an output; determining from the voice data a current mood and/or cognitive state of the user; determining an output in response to the request; and adjusting the output and/or the form of the output according to the determined current mood and/or cognitive state of the user.

2. A method according to aspect 1, wherein determining from the voice data a current mood and/or cognitive state of the user comprises converting the voice data to text data and analyzing the syntax and word choice associated with the text data.

3. A method according to either aspect 1 or aspect 2, wherein determining from the voice data a current mood and/or cognitive state of the user comprises analyzing acoustic features of the voice data.

4. A method according to any one of aspects 1 to 3, wherein determining from the voice data a current mood and/or cognitive state of the user comprises executing one or more machine learning algorithms using as input to the one or more machine learning algorithms syntax, word choice, and/or acoustic features of the voice data.

5. A method according to aspect 4, wherein determining from the voice data a current mood and/or cognitive state of the user comprises using as input to the one or more machine learning algorithms historical data associated with the user.

6. A method according to either aspect 4 or aspect 5, wherein determining from the voice data a current mood and/or cognitive state of the user comprises using as input to the one or more machine learning algorithms data associated with a population of users.

7. A method according to aspect 6, wherein the population of users share a common medical condition with the user.

8. A method according to aspect 7, wherein the common medical condition is diabetes.

9. A method according to any one of aspects 1 to 8, wherein determining an output in response to the request comprises retrieving information from a best practices medical database.

10. A method according to any one of aspects 1 to 9, wherein determining an output in response to the request comprises retrieving information from an electronic medical record associated with the user.

11. A method according to any one of aspects 1 to 10, wherein determining an output in response to the request comprises retrieving information from a medication management module.

12. A method according to any one of aspects 1 to 11, wherein determining an output in response to the request comprises retrieving information from a source of nutrition data.

13. A method comprising: receiving data representative of an input from a user; processing the data to determine one or more sentiment features for the data; processing the data to determine one or more request characteristics corresponding to the data; processing the one or more sentiment features to (1) identify a domain-specific service module indicated by the one or more sentiment features, (2) identify a request indicated by the one or more sentiment features, and (3) output a structured representation of the identified request; transmitting to the identified domain-specific service module the structured representation of the identified request; transmitting to the identified domain-specific service module the identified request characteristics; determining, in the domain-specific service module, from the one or more request characteristics and/or the identified request, one or more real-time user characteristics; determining in the domain-specific service module a response to the request indicated by the one or more sentiment features based on the structured representation of the identified request and the identified real-time user characteristics, the response represented as text; processing the response to output a speech representation of the text response; and transmitting the speech representation to a voice-enabled device.

14. A method according to aspect 13, wherein the data received are voice stream data received from a voice-enabled device and are representative of a voice input from the user.

15. A method according to aspect 13 or aspect 14, wherein the one or more real-time user characteristics comprise either a mood of the user, a cognitive state of the user, or both.

16. A method according to either aspect 14 or aspect 15, wherein processing the voice stream data to determine one or more request characteristics corresponding to the voice stream data comprises processing a text-based representation of the voice stream data to determine syntax and/or word choice of the text-based representation.

17. A method according to any one of aspects 14 to 16, wherein processing the voice stream data to determine one or more request characteristics corresponding to the voice stream data comprises analyzing acoustic features of the voice stream.

18. A method according to aspect 17, wherein the acoustic features include one or more of the group consisting of: speech volume, speech speed, speech clarity, timbre of the voice, vocal inflections, and vocal pitch.

19. A method according to any one of aspects 13 to 18, wherein processing the one or more sentiment features to identify a domain-specific service indicated by the one or more sentiment features comprises selecting a domain-specific service based on a combination of sentiment features.

20. A method according to any one of aspects 13 to 18, wherein processing the one or more sentiment features to identify a domain-specific service indicated by the one or more sentiment features comprises selecting a domain-specific service based on a keyword corresponding specifically to the domain-specific service.

21. A method according to any one of aspects 13 to 20, wherein determining in the domain-specific service module a response to the request comprises identifying a third-party resource from which to retrieve information.

22. A method according to aspect 21, wherein determining in the domain-specific service module a response to the request comprises invoking an application programming interface (API) to access the third-party resource.

23. A method according to any one of aspects 13 to 22, wherein determining in the domain specific service module one or more real-time user characteristics comprises using a machine learning algorithm to analyze the one or more request characteristics.

24. A method according to aspect 23, wherein analyzing the one or more request characteristics using a machine learning algorithm comprises analyzing a database storing machine-learning data related to the user and/or to a set of users having in common with the user a particular medical condition.

25. A method according to either aspect 23 or aspect 24, wherein analyzing the one or more request characteristics using a machine learning algorithm comprises determining from the analysis whether to update a database storing machine-learning data.

26. A method according to any one of aspects 13 to 25, wherein determining a response to the request comprises selecting information to include in the response based on a determined mood or cognitive state of the user.

27. A method according to any one of aspects 13 to 26, wherein determining a response to the request comprises adjust the language used in the response based on a determined mood or cognitive state of the user.

28. A method according to any one of aspects 13 to 27, further comprising receiving at the domain specific service module additional data related to the request, the additional data including data from a best practices medical database, and analyzing in the domain-specific service module the request and the additional data to determine the response.

29. A method according to any one of aspects 13 to 28, further comprising receiving at the domain specific service module additional data related to the request, the additional data including data from an electronic medical record associated with the user, and analyzing in the domain-specific service module the request and the additional data to determine the response.

30. A method according to any one of aspects 13 to 29, further comprising receiving at the domain specific service module additional data related to the request, the additional data including data from medication management module, and analyzing in the domain-specific service module the request and the additional data to determine the response.

31. A method according to any one of aspects 13 to 30, further comprising receiving input data from one or more sources selected from the group consisting of: a device recording physiological data of the user; a device monitoring health status of the user; and a mobile device associated with the user and providing environmental data and/or data related to the user's interaction with the mobile device.

32. A method according to any one of aspects 13 to 31, further comprising receiving input data from a mobile device associated with the user and providing environmental data and/or data related to the user's interaction with the mobile device.

33. A method according to aspect 32, wherein the mobile device is providing data related to the user's interaction with the mobile device, and the data related to the user's interaction with the mobile device comprises keyboard metadata.

34. A method according to aspect 33, further comprising analyzing the keyboard metadata using a machine learning algorithm to determine a mood and/or cognitive state of the user.

35. A method according to aspect 34, wherein analyzing the keyboard metadata using a machine learning algorithm comprises analyzing the keyboard metadata and historical keyboard metadata to determine a mood and/or cognitive state of the user.

36. A method according to either aspect 34 or aspect 35, wherein analyzing the keyboard metadata using a machine learning algorithm comprises analyzing the keyboard metadata and real-time user characteristics to determine the mood and/or cognitive state of the user.

37. A method according to any one of aspects 14 to 36, wherein the voice-enabled device is a mobile device.

38. A method according to any one of aspects 13 to 37, wherein the domain-specific service module is configured to provide information and services to users having a specific medical condition.

39. A method according to aspect 38, wherein the specific medical condition is diabetes.

40. A method according to any one of aspects 13 to 39, wherein receiving data representative of an input from a user comprises receiving data from one or more of the group consisting of: a device recording physiological data of the user, a smart watch, a fitness tracker, a medical device, a blood glucose monitoring device, and a mobile device associated with the user.

41. A system comprising: a server comprising a processor configured to execute machine readable instructions, the machine readable instructions causing the processor to: receive input data from a plurality of selectively connected input sources, the input sources including at least one of the group consisting of: a device recording physiological data of a user; a device monitoring health status of the user; a mobile device associated with the user and providing environmental data and/or data related to the user's interaction with the mobile device; a source of electronic medical record data; and a device receiving voice input from the user; analyze the received input data relative to prior data stored in a machine-learning algorithm database (MLAD), using one or more machine learning algorithms to determine, within a predetermined time frame, (1) a notification to send to the user and/or (2) an update to the MLAD, wherein the determination of the notification and/or the determination of the update to the MLAD is based on input data received during the predetermined time frame, data in the MLAD, and data stored in a best practices medical database.

42. A system according to aspect 41, wherein the input source includes a device recording physiological data of the user.

43. A system according to aspect 42, wherein the device recording physiological data of the user comprises a smart watch.

44. A system according to aspect 42, wherein the device recording physiological data of the user comprises a fitness tracker.

45. A system according to aspect 42, wherein the device recording physiological data of the user comprises a medical device.

46. A system according to aspect 42, wherein the medical device comprises a blood glucose monitoring device.

47. A system according to any one of aspects 41 to 46, wherein the input source includes a mobile device associated with the user.

48. A system according to aspect 47, wherein the mobile device comprises an application that analyzes user interaction with a software keyboard and generates metadata corresponding to the user interaction with the software keyboard.

49. A system according to aspect 48, wherein the metadata corresponding to the user interaction with the software keyboard comprises one or more of the group consisting of: session length, average session length, interkey delay, average interkey delay, keypress duration, average keypress duration, distance between consecutive keys, ratio of interkey delay to distance, spacebar ratio, backspace ratio, autocorrect ratio, circadian baseline similarity, and metadata feature variability.

50. A system according to any one of aspects 41 to 49, wherein the notification to send to the user is a reminder to take a medication.

51. A system according to any one of aspects 41 to 49, wherein the notification to send to the user is a reminder of an appointment.

52. A system according to any one of aspects 41 to 49, wherein the notification to send to the user is a response to a request made by the user.

53. A system according to aspect 52, wherein the response to the request made by the user includes providing information from the best practices medical database.

54. A system according to any one of aspects 41 to 53, wherein the update to the MLAD is an update to the prior data stored in the MLAD.

55. A system according to aspect 54, wherein the prior data stored in the MLAD include data of the mood and/or cognitive state of the user.

56. A system according to either aspect 54 or aspect 55, wherein the prior data stored in the MLAD include metadata of acoustic or sentiment information associated with a request by a user made using a voice-enabled assistant.

57. A system according to any one of aspects 54 to 56, wherein the prior data stored in the MLAD include keyboard metadata received from a mobile device.

58. A method comprising: receiving at a server, a request from a user for information or to perform an action; receiving at the server additional data related to the request, the additional data including at least one of the group consisting of: (1) data from a best practices medical database; (2) real-time data indicative of a mood of the user and/or a cognitive state of the user; and (3) a database storing machine-learning data related to the user and/or a set of users having in common with the user a particular characteristic; analyzing in the server the request and the additional data related to the request to generate the information requested by the user or perform the action requested by the user.

59. A method according to aspect 58, wherein the request is in the form of a voice request made by the user.

60. A method according to aspect 58, wherein the request is in the form of a typed request.

61. A method according to any one of aspects 58 to 60, wherein the additional data include real-time data indicative of the mood of the user and/or the cognitive state of the user, and wherein the additional data include metadata related to the syntax or word choice in the request.

62. A method according to aspect 61, wherein the additional data further includes the database storing machine-learning data, and wherein analyzing the request and additional data includes using one or more machine learning algorithms to determine, based on the machine-learning data related to the user and/or the set of users having in common with the user a particular characteristic.

63. A method according to aspect 62, wherein the particular characteristic is a medical condition.

64. A method according to aspect 63, wherein the common medical condition is diabetes.

65. A method of selectively, and in real time, generating medical notifications for delivery to a device associated with a patient, the method comprising: receiving, at a server, data associated with the patient; analyzing (1) the received data, (2) a best practices medical database, and (3) a database storing machine-learning data related to the patient and/or a set of users having in common with the patient a particular medical condition; determining from the analysis whether to generate a medical notification and transmit the medical notification to the device associated with the patient; and determining from the analysis whether to update the database.

1-67. (canceled)

68. A method comprising:
receiving metadata associated with a user's interaction with a mobile device;
analyzing the received metadata by comparing the received metadata to previously-received metadata;
determining, from the received metadata and the previously-received metadata, a current mood and/or cognitive state of the user;
outputting the current mood and/or cognitive state of the user.

69. A method according to claim 68, wherein analyzing the received metadata by comparing the received metadata to previously received metadata comprises inputting into a machine learning algorithm the received metadata, and wherein the machine learning algorithm determines and outputs the current mood and/or cognitive state of the user.

70. A method according to claim 68, further comprising comparing the current mood and/or cognitive state of the user to a previously stored mood and/or cognitive state of a user to determine a change in the mood and/or cognitive state of the user.

71. A method according to claim 68, wherein receiving metadata associated with a user's interaction with a mobile device comprises receiving keyboard metadata.

72. A method according to claim 71, wherein receiving keyboard metadata comprises receiving one or more of the group consisting of: session length, average session length, interkey delay, average interkey delay, keypress duration, average keypress duration, distance between consecutive keys, ratio of interkey delay to distance, spacebar ratio, backspace ratio, autocorrect ratio, circadian baseline similarity, and metadata feature variability.

73. A method according to claim 68, wherein receiving metadata associated with a user's interaction with a mobile device comprises receiving voice metadata.

74. A method according to claim 6, wherein the voice metadata is selected from the group consisting of: speech volume, speech speed, presence of slurring, degree of slurring, speech clarity, timbre of the voice, vocal inflections, and vocal pitch.

75. A method according to claim 68, wherein the method is performed on the mobile device.

76. A method according to claim 68, wherein the received metadata are generated by an application, executing on the mobile device, that analyzes the user's interaction with a software keyboard and generates metadata corresponding to the user interaction with the software keyboard.

77. A method according to claim 68, wherein receiving metadata associated with a user's interaction with a mobile device comprises receiving accelerometer metadata.

78. A method according to claim 68, further comprising determining a notification according to the determined current mood and/or cognitive state.

79. A method according to claim 78, wherein the notification is one of the group consisting of: a reminder to take a medication, a reminder of an appointment, and a response to a request made by the user.

80. A method according to claim 68, wherein the previously-received metadata are associated with the user's previous interaction with the mobile device.

81. A method according to claim 68, further comprising storing in a database the received metadata and/or the determined current mood and/or cognitive state of the user.

82. A method according to claim 81, wherein:
analyzing the received metadata by comparing the received metadata to previously received metadata comprises inputting into a machine learning algorithm the received metadata,
the machine learning algorithm determines and outputs the current mood and/or cognitive state of the user, and
the stored metadata and/or the stored determined current mood and/or cognitive state of the user are used to update the machine learning algorithm.

83. A method according to claim 68, wherein analyzing the received metadata by comparing the received metadata to previously-received metadata comprises analyzing the received metadata by comparing the received metadata to previously-received metadata associated with a population of users sharing a common characteristic with the user.

84. A method according to claim 83, wherein the common characteristic is a medical condition.

85. A method according to claim 84, wherein the medical condition is diabetes or depression.

86. A method comprising:
receiving from a voice-enabled device associated with a user, voice data, the voice data indicating a request for an output;
determining from the voice data a current mood and/or cognitive state of the user;
determining an output in response to the request; and
adjusting a content of the output and/or a form of the output according to the determined current mood and/or cognitive state of the user.

87. A method according to claim 86, wherein the voice data is selected from the group consisting of: speech volume, speech speed, presence of slurring, degree of slurring, speech clarity, timbre of the voice, vocal inflections, and vocal pitch.

88. A method comprising:
receiving data representative of an input from a user;
processing the data to determine one or more sentiment features for the data;
processing the data to determine one or more request characteristics corresponding to the data;
processing the one or more sentiment features to (1) identify a domain-specific service module indicated by the one or more sentiment features, (2) identify a request indicated by the one or more sentiment features, and (3) output a structure representation of the identified request;
transmitting to the identified domain-specific service module the structured representation of the identified request;
transmitting to the identified domain-specific service module the identified request characteristics;
determining, in the domain-specific service module, from the one or more request characteristics and/or the identified request, one or more real-time user characteristics;
determining in the domain-specific service module a response to the request indicated by the one or more sentiment features based on the structured representation of the identified request and the identified real-time user characteristics, the response represented as text;
processing the response to output a speech representation of the text response; and
transmitting the speech representation to a voice-enabled device.
